(19) United States
(12) Patent Application Publication
(10) Pub. No.: US 2007/0110727 A1
(43) Pub. Date: May 17, 2007

(54) Multipotent stem cells isolated from umbilical cord blood and the cellular therapeutic agent comprising the same for treating ischemic disease

(76) Inventor: Kyung Sun Kang, Seoul (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/555,852

(22) PCT Filed: Aug. 26, 2005
(86) PCT No.: PCT/KR05/02834

§ 371 Date: Nov. 7, 2005

(51) Int. Cl. C12N 5/08; A61K 35/36

(52) U.S. Cl. 424/93.7; 435/371

(57) ABSTRACT

The present invention relates to multipotent stem cells, adult stem cells isolated by culturing umbilical cord blood in a medium containing human serum or plasma, and a cellular therapeutic agent for ischemic necrosis resulting from occlusive arterial disease, which contains the multipotent stem cells as active ingredients. The multipotent stem cells according to the invention have ability to differentiate into osteogenic cells or nerve cells, even if they are adult stem cells. Thus, the multipotent stem cells will be useful for the treatment of not only diseases where the stagnation of femoral arterial circulation causes peripheral circulatory disorders to degrade microvascular tissue, leading to ischemic necrosis, but also ischemic diseases, such as cerebral infarction, myocardial infarction and the necrosis of limb ends resulting from diabetic sequelae.

### TECHNICAL FIELD

The present invention relates to multipotent stem cells, adult stem cells isolated by culturing umbilical cord blood in a medium containing human serum or plasma, and a cellular therapeutic agent for ischemic necrosis resulting from occlusive arterial disease, which contains the multipotent stem cells as active ingredients.

### BACKGROUND ART

With the aging of society, the case of blood circulatory disorder caused by occlusive arterial disease increases. Typical diseases with this blood circulatory disorder include myocardial infarction, cerebral infraction, Buerger's disease which is occlusive peripheral vascular disease where blood vessels contract or become narrower due to inflammation or vascular wall changes in small arteries or veins at hands or feet, mainly below knees, so that blood vessels are occluded entirely, as well as occlusive arterial disease caused by microvascular or macrovascular disorders resulting from diabetic complication.

Myocardial infarction is a disease where a portion of a coronary artery supplying oxygen and nutrients to the heart is occluded to stop blood circulation so that a cardiac wall (i.e., myocardium) at that portion decays. Cerebral infractions include thromotic cerebral infraction where blood vessels become narrower or are occluded due to atherosclerosis to make blood flow and then oxygen insufficient so that cerebral tissue is killed, and embolic cerebral infarction where a piece (embolus) detached from the thrombus of an artery with atherosclerosis blocks arteries during its flowing through cerebral blood vessels so that cerebral tissue downstream thereof is killed.

Buerger's disease is inferred to result from the complex action of various factors, although its direct cause was not found. Age, sex, family genetic factors, autoimmunity, professions, smoking and the like are considered as secondary causative factors. Also, because patients with Buerger's disease have high blood fibrinogen levels, the constriction of peripheral blood vessels resulting from the overcoagulation of blood, the overaggregation of platelets or the oversensitiveness of the sympathetic nerve is presumed to be a causative factor.

Gangrene of extremities, which is one of the most terrible symptoms in diabetic complication, occurs in heavy diabetes and is a hazardous symptom where hand or foot ends decay in black. Although its exact cause cannot be understood, it is inferred to result from symptoms, such as external wounds, burns and purulence. The gangrene of extremities is a disease which frequently occurs in over 50 years-old patients and involves inflammation, blisters, ulcers, fever and the like, and in severe cases, leads to the cutting of hands and feet or death.

This occlusive arterial disease is higher in frequency in white men and assumed to account for about 15% of total peripheral arterial diseases, although its authentic statistical data in Korea is not yet sufficient (Laohapensang, K. et al., Eur. J. Vasc. Endovasc. Surg., 28(4):418, 2004). The occlusive arterial disease invades small or middle-sized arteries and veins at the distal upper and lower limbs, and the stagnation of arterial circulation at the end of limbs mostly causes peripheral circulatory disorders to break down the tissue of microvascular tissue, resulting in ischemic disease. In this ischemic necrosis, due to the reduction of arterial blood flow pressure and the stagnation of capillary blood flow, leucocytes and platelets are activated and vascular epithelium gets damages, resulting in local hypoxia and metabolic changes.

Therapies for occlusive arterial disease include arterial bypass surgery where artificial blood vessels are used to make new blood vessels between the upper and lower portions of occluded arteries such that blood can flow well through the arteries, as well as drug therapy of administering vasodilators, and a therapeutic method of anesthetizing the sympathetic nerve of the lumbar so as to enlarge small blood vessels at limbs. Recently, studies for the development of a therapeutic method with the use of vascular endothelial growth factor 165 (VEGF 165) (Kim, H. J. et al., Exp. Mol. Med., 36(4):336, 2004) and the development of a therapeutic method by autologous hematopoietic cells transplantation (Miyamoto, M. et al., Cell Transplant., 13(4):429, 2004).

The ischemic necrosis is not yet found for its exact cause and has a characteristic in that the disease continues to progress, it is in a stage where a sure treatment method therefor is not yet established. A final purpose of treating ischemic necrosis caused by occlusive arterial diseases, such as Buerger's disease, is to make arterial blood flow smooth.

Meanwhile, the term “stem cells” refers to cells having not only self-replication ability but also the ability to differentiate into at least two cells, and can be divided into totipotent stem cells, pluripotent stem cells, and multipotent stem cells.

Totipotent stem cells are cells having totipotent properties capable of developing into one perfect individual, and these properties are possessed by cells up to the 8-cell stage after the fertilization of an oocyte and a sperm. When these cells are isolated and transplanted into the uterus, they can develop into one perfect individual.

Pluripotent stem cells, which are cells capable of developing into various cells and tissues derived from the ectodermal, mesodermal and endoderaml layers, are derived from an inner cell mass located inside of blastocysts at 4-5 days after fertilization. These cells are called “embryonic stem cells” and can differentiate into various other tissue cells but not form new living organisms.

Multipotent stem cells, which are stem cells capable of differentiating into only cells specific to tissues and organs containing these cells, are involved not only in the growth and development of various tissues and organs in the fetal, neonatal and adult periods but also in the maintenance of homeostasis of adult tissue and in function to induce regeneration upon tissue damage. Tissue-specific multipotent cells are collectively called “adult stem cells”.

Umbilical cord blood-derived adult stem cells known up to now include mesenchymal stem cells capable of differentiating into osteocytes or skeleton muscles (Lee, O. K. et al., Blood, 103:1669, 2004; Gang, E. J. et al., BBRC, 321:102, 2004; Gang, E. J. et al., Stem Cell, 22:617, 2004), heart stem cells capable of differentiating into heart cells (US 2004/0126879) and endothelial progenitor cells (Yamamoto, K. et al., Arterio. Thromb. Vasc. Biol., 24:192, 2004).

However, because the stem cells show the ability of differentiation into limited tissues, including differentiation into osteocytes or skeleton muscles by mesenchymal stem cells, differentiation into heart cells by heart stem cells, and differentiation into vascular endothelial cells by endothelial progenitor cells, these cells are difficult to define as true multipotent stem cells. Also, in the above-cited prior literatures, an FBS-containing medium was used in a process of isolating stem cells, and thus, the number and kind of obtainable cells would be unavoidably limited.

Accordingly, the present inventors have cultured umbilical cord-derived blood using human serum or plasma as a medium, and as a result, found that the resulting adult cells are excellent in the adhesion ability to plastics, differentiate into various tissues, such as osteogenic cells and nerve cells, and are useful as a cellular therapeutic agent for ischemic necrosis caused by occlusive arterial disease, thereby completing the present invention.

### DISCLOSURE OF THE INVENTION

It is a main object of the present invention to provide multipotent stem cells, adult stem cells derived from umbilical cord blood, and a production method thereof.

Another object of the present invention is to provide a cellular therapeutic agent for ischemic necrosis caused by occlusive arterial disease, which contains the adult stem cells as active ingredients.

To achieve the above objects, in one aspect, the present invention provides adult stem cells which are obtained by culturing umbilical cord-derived blood in a medium containing 5-20% of human serum or plasma and show the characteristics of:
(a) showing positive immunological responses to all of CD24, CD29, CD31, CD33, CD45 and CD49B, and negative immunological responses to CD34, CD51/61, CD62L, CD62P, CD73, CD90, CD133 and CD135;
(b) growing adhered to plastics and showing round-shaped or spindle-shaped morphological features; and
(c) having ability to differentiate into mesodermal, endodermal and ectodermal cells.

In still another aspect, the present invention provides a method for producing adult stem cells showing the characteristics (a) to (c), the method comprising the steps of: culturing umbilical cord-derived blood in a medium containing 5-20% of human serum or plasma; and recovering the adult stem cells.

In the present invention, the adult stem cells additionally show positive immunological responses to SH-2 and/or SH-3, and positive or negative immunological responses to CD44, CD105 and CD117. Also, the mesodermal cells are osteogenic cells, nerve cells or endothelial cells.

In yet another aspect, the present invention provides a cellular therapeutic agent for ischemic necrotic disease caused by occlusive arterial disease, which contains the adult stem cells as active ingredients. The ischemic necrotic disease is myocardial infarction, cerebral infarction, Buerger's disease, and gangrene of extremities resulting from diabetic complications.

The above and other objects, features and embodiments of the present invention will be more clearly understood from the following detailed description and the accompanying claims.

### BRIEF DESCRIPTION OF DRAWINGS

FIG. 1 illustrates photographs showing the comparison of cell adhesion between cells obtained by culturing umbilical cord blood in FBS and cells obtained by culturing umbilical cord cells in human plasma-containing medium. Left photographs in FIG. 1 were taken at 200× magnification and right photographs were taken at 400× magnification.

FIGS. 2A and 2B show that multipotent stem cells derived from umbilical cord blood differentiated into osteogenic cells, and FIGS. 2C and 2D show the results of Von-Kossa staining.

FIG. 3 shows the binding between umbilical cord blood-derived multipotent stem cells and specific antigens. FIGS. 3A and 3B illustrate that the multipotent stem cells show positive responses to NSE (neuron-specific enolse), a neuron-specific antigen, and GFAP (glial fibrillary acidic protein), an astrocyte-specific antigen, respectively, and C and D show control groups.

FIG. 4 shows the immunological characteristics of umbilical cord blood-derived multipotent stem cells, measured by flow cytometry. A and E: control groups; B: CD34; C: CD45; D: SH-2; and F: SH-3.

FIG. 5 shows the results of PAS staining conducted to examine the expression or non-expression of antigens in the inventive adult stem cells.

FIG. 6 shows results for a control mouse group with ischemic necrosis, which was not administered with the inventive stem cells at 30 days after vascular severance, in which a red circle shows a region where an amputation event occurred.

FIG. 7 shows results for a mouse group with ischemic necrosis, which was administered with the inventive stem cells immediately at 30 days after vascular severance, in which a red circle shows a region where an amputation event occurred.

FIG. 8 shows results for a mouse group with ischemic necrosis, which was administered with the inventive stem cells at 31 days after vascular severance, in which a red circle shows a region where an amputation event occurred.

FIG. 9 is a graphic diagram showing symptom days and amputation days in a mouse model with ischemic necrosis.

FIG. 10 is a graphic diagram showing the amputation rate of each group in a mouse model with ischemic necrosis.

FIG. 11 illustrates X-ray photographs taken by angiography at 30 days after vascular severance for each group in a mouse model with ischemic necrosis.

FIG. 12 shows that femoral muscle tissues sectioned after the autopsy of a mouse model with ischemic necrosis are traced with a human-specific probe by an in situ hybridization technique.

### DETAILED DESCRIPTION OF THE INVENTION

The present invention relates to multipotent stem cells isolated from umbilical cord blood.

As used herein, the term “umbilical cord blood” is defined as blood collected from umbilical veins connecting the placenta with an embryo. The multipotent stem cells according to the present invention are preferably derived from human umbilical blood.

Although a method for isolating and purifying multipotent stem cells from umbilical cord blood is not specifically limited, the following method can be used. First, blood collected from umbilical blood is diluted with PBS at a given ratio before stirring, and the stirred solution is separated on ficoll at a ratio of 10-15:20-30, and preferably 15:25. For this purpose, to 10-20 ml and preferably 15 ml of a ficoll solution, the above sample solution spills smoothly to cause layer separation, followed by centrifugation. Then, once three different layers are formed, a buffer coat of the middle layer is taken by a micropipette, washed with 2-5 times with HBSS, and centrifuged to collect an umbilical cord blood-derived multipotent stem cell solution.

Pellets finally obtained by centrifugation are diluted in 1-5 ml of medium (MSCBM+MSCGM (Cambrex, USA) cell culture solution) containing 5-15% of autologous or allologous human serum or plasma and 10 ng/ml of alkaline fibroblast growth factor (bFGF)), and cultured on a flask. In the case of seeding into a 75-flask, about 10⁶-10⁸ cells are placed to which 5-15% of autologous or allologous human serum and 10 ng/ml of alkaline fibroblast growth factor (bFGF) are added. The cells are cultured in a 5% CO₂ incubator at 37° C. The cells first seeded into the flask are transferred after about 1-2 days. The medium is replaced one time at about 3-7-day intervals.

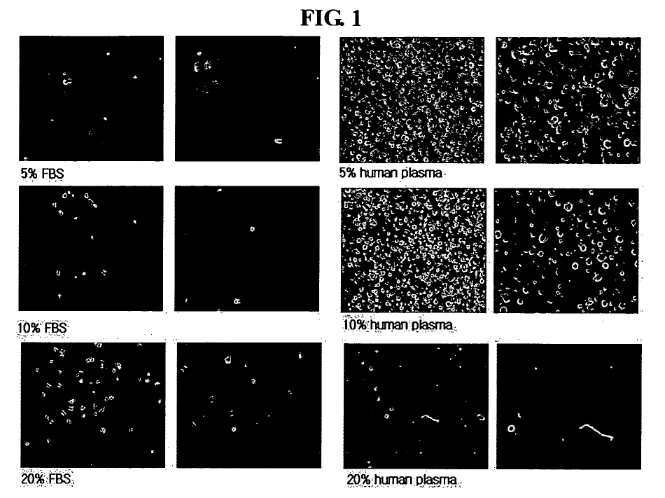

In the present invention, the umbilical cord blood-derived multipotent stem cell solution was cultured in a human serum or plasma-containing medium in place of the existing FBS. As a result, as shown in FIG. 1, the number of cells growing adhered to a culture dish was much larger in media containing 5% and 10% human plasma than in media containing 5% FBS and 10% FBS.

Methods of obtaining multipotent stem cells expressing the desired surface antigens from the umbilical cord blood-derived stem cell solution obtained above include a FACS method using a flow cytometer with sorting function (Int. Immunol., 10(3):275, 1998), a method using magnetic beads, and a panning method using an antibody specifically recognizing multipotent stem cells (J. Immunol., 141(8):2797, 1998). Also, methods for obtaining multipotent stem cells from a large amount of culture solution include a method where antibodies specifically recognizing molecules expressed on the surface of cells (hereinafter, referred to as “surface antigens”) are used alone or in combination as columns.

Flow cytometry sorting methods may include a water drop charge method and a cell capture method. In any of these methods, an antibody specifically recognizing a antigen on the cell surface is fluorescently labeled, the intensity of fluorescence emitted from an antibody bonded to the molecule expressed on the surface of the cell is converted to an electric signal, whereby the expressed amount of the antigen can be quantified. It is also possible to separate cells expressing a plurality of surface antigens by combination of fluorescence types used therefor. Examples of a fluorescence which can be used in this case include FITC (fluorescein isothiocyanate), PE (phycoerythrin), APC (allo-phycocyanin), TR (Texas Red), Cy 3, CyChrome, Red 613, Red 670, PerCP, TRI-Color, Quantum Red, etc.

FACS methods using a flow cytometer, which can be used in the present invention, include: a method where the above stem cell solution is collected, from which cells are isolated by, for example, centrifugation, and stained directly with antibodies; and a method where the cells are cultured and grown in a suitable and then stained with antibodies. The staining of cells is performed by mixing a primary antibody recognizing a surface antigen with the target cell sample and incubating the mixture on ice for 30 minutes to 1 hour. When the primary antibody is fluorescently labeled, the cells are isolated with a flow cytometer after washing. When the primary antibody is not fluorescently labeled, cells showing the reaction between the primary antibody and a secondary antibody having binding activity to the primary antibody are washed, mixed with each other, and incubated on ice water for 30 minutes to 1 hour. After washing, the cells stained with the primary and secondary antibodies are isolated with a flow cytometer.

The method using magnetic beads allows the mass isolation of cells expressing the target surface antigens. Although this method is lower in isolated cell purity than the above-described method using the flow cytometer, it can secure sufficiently cell purity by repeated purification.

Surface antigens may include hematopoietic antigens, surface antigens of mesenchymal cells, and neuron-specific antigens. The hematopoietic antigens include CD34 and CD45, and the surface antigens of mesenchymal cells include SH-2 and SH-3, and the neuron-specific antigens NSE and GFAP. The single or combined use of antigens recognizing the above-described surface antigens allows the desired cells to be obtained.

In the present invention, the umbilical cord blood-derived multipotent stem cells obtained above were transplanted into a mouse model with ischemic necrosis, and as a result, it could be seen that, in cases administered with the inventive multipotent stem cells, the induction of necrotic symptoms was inhibited, and new blood vessels substituting for severed femoral arteries were produced.

Accordingly, the inventive multipotent stem cells can be used as a cellular therapeutic agent for ischemic necrotic disease caused by occlusive arterial disease. The cellular therapeutic agent for ischemic necrotic disease preferably contains a high purity of cells having differentiation ability into vascular cells, among the inventive multipotent stem cells.

The inventive multipotent stem cells are allowed to differentiate and proliferate in vitro depending on the size and site of ischemic necrosis, to obtain cells having differentiation ability into the desired cells which can be used for the treatment of ischemic necrotic disease by transplantation into ischemic necrotic sites. Also, the inventive multipotent stem cells may be transplanted directly into ischemic necrotic sites.

The inventive multipotent stem cells show positive responses to monocyte-macrophage antigen CD45, and negative responses to hematopoietic lineage antigen CD34, and thus, are considered as stem cells which are in differentiation from hematopoietic cells into monocytes.

### EXAMPLES

Hereinafter, the present invention will be described in more detail by examples. It is to be understood, however, that these examples are for illustrative purpose only and are not construed to limit the scope of the present invention.

### Example 1

### Isolation of Multipotent Stem Cells from Umbilical Cord Blood

Umbilical cord blood was collected from full-term and preterm newborns in Seoul National University Hospital and Samsung Cheil Hospital according to Institutional Review Board guidelines.

70-100 ml of a blood sample taken from the collected umbilical cord blood was diluted with PBS at a ratio of 1:1 followed by stirring. Then, the blood sample was separated on ficoll at a ratio of 15:25, in which the blood sample spilled smoothly onto 15 ml of picoll solution to cause layer separation, followed by centrifugation at 2500 rpm for 20 minutes. After the centrifugation, three different layers were formed, and among them, a buffer coat of the middle layer was taken with a micropipette, washed three times with HBSS, followed by centrifugation at 1500 rpm for 15 minutes, thus obtaining pellets (umbilical cord blood-derived multipotent stem cell solution).

### Example 2

### Differentiation of Umbilical Cord Blood-Derived Multipotent Stem Cells into Osteogenic Cells

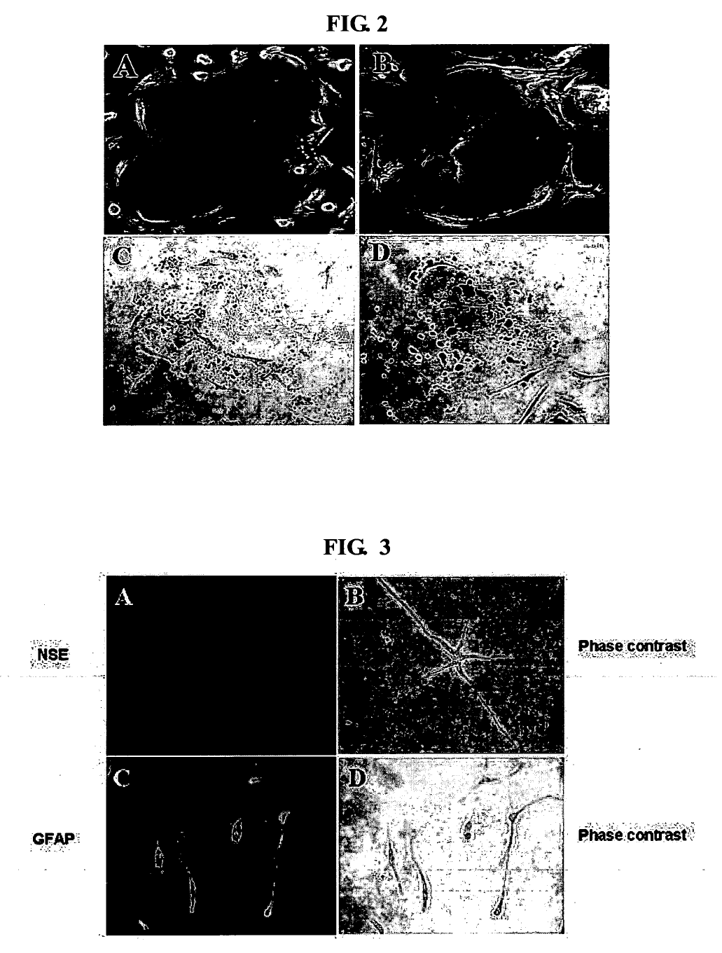

The umbilical cord blood-derived multipotent stem cell solution obtained in Example 1 was diluted in 1 ml of osteogenesis inducing medium (0.1 μmol/L dexamethasone (Sigma, USA), 0.05 mmol/L ascorbic acid-2-phosphate (Sigma, USA) and 10 mmol/L beta-glycophosphate (Sigma), 5-20% human serum or plasma) and counted for cell number. Then, the cell solution was cultured in a flask (5% CO₂; 37° C.; medium replaced one time at 3-4-day intervals) to induce the differentiation of the multipotent stem cells into osteogenic cells. 14 days after the initiation of the culture, it was confirmed by Von-Kassa staining that the umbilical cord blood-derived multipotent stem cells differentiated into osteogenic cells (see FIG. 2).

### Example 3

### Differentiation of Umbilical Cord Blood-Derived Multipotent Stem Cells into Nerve Cells

The umbilical cord blood-derived multipotent stem cell solution obtained in Example 1 were diluted in 1 ml of nerve forming medium (containing 10 ng/ml basic fibroblast growth (Roche, Switzerland), 10 ng/ml human epidermal growth factor (Roche, Switzerland) and 10 ng/ml human neural growth factor (Invitrogen, USA) in 5-20% human serum or plasma) and counted for cell number. The cell solution was cultured on a flask at 5% CO₂ and 37° C. to induce differentiation from the multipotent stem cells into nerve cells. At 14 days after the initiation of the culture, it was confirmed that the multipotent stem cells showed positive responses to NSE (neuron-specific enolase), a neuron-specific antigen, and GFAP (glial fibrillary acidic protein), an astrocyte-specific antigen, indicating that the umbilical cord blood-derived multipotent stem cells differentiated into nerve cells (see FIG. 3).

### Example 4

### Immunological Characteristics of Umbilical Cord Blood-Derived Multipotent Stem Cells

To examine the immunological characteristics of the umbilical cord blood-derived multipotent stem cells obtained in Example 1, the expression pattern of cell-surface antigens was analyzed. For this purpose, 2×10⁶-10⁷ of the cells cultured in Example 1 were washed with PBS solution and incubated with antibodies to the respective antigens at room temperature. The expression or non-expression of the antigens was analyzed with a flow cytometer. Also, PAS (periodic acid staining) was conducted.

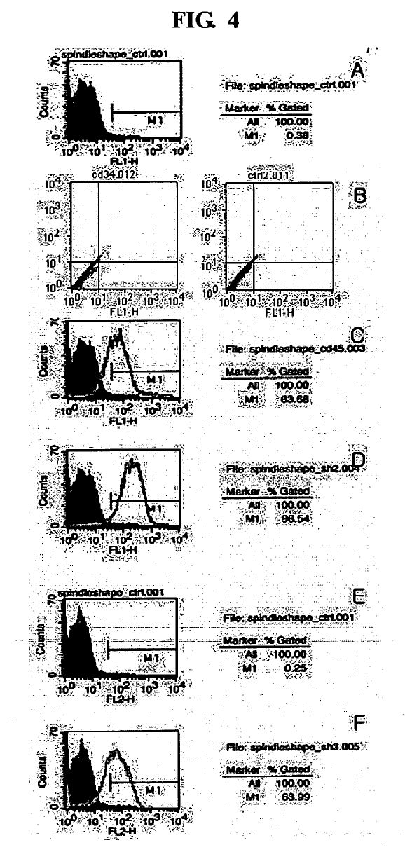

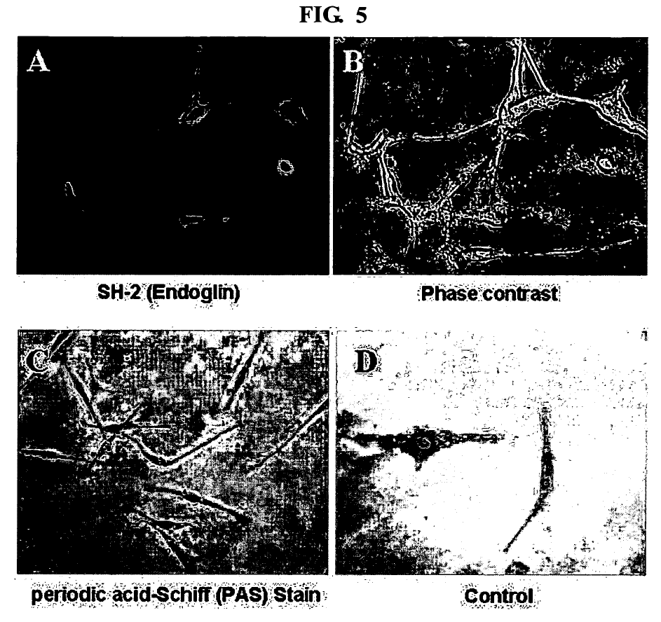

As a result, as shown in FIG. 4, the inventive umbilical cord blood-derived multipotent stem cells showed positive responses of 63.38%, 96.54% and 63.99% to CD45, SH-2 and SH-3, respectively, and a negative response of more than 90% to CD34. Also, immunophenotypes to other antigens were analyzed and as a result, the multipotent stem cells showed immunological characteristics of CD51/61-negative, CD62L-negative, CD62P-negative, CD133-negative, CD135-negative, CD90-negative, CD29-positive, CD44-positive or negative, CD49B-positive, CD 105(SH-2)-positive or negative, and CD90-negative phenotypes.

Meanwhile, as shown in FIG. 5, the multipotent stem cells showed positive responses in PAS staining.

### Example 5

### Therapeutic Effect of Multipotent Stem Cells on Ischemic Necrotic Model

(1) Preparation of Test Animals

Twenty 6-week-old male BALB/cANCrjBgi-nu mice were purchased from Orient Co., Ltd. and acclimated for one week. Then, among them, 18 animals were selected for use in test examples below. The selected animals were raised on an MI rack mounted with a HEPA filter at a temperature of 32±3° C., a relative humidity of 60±10%, ventilations of 10-12/hr, a lighting time of 12 hrs, and an illumination intensity of 250-200 lux while allowing to freely taking solid feed (Purina) sterilized with steam under high pressure and drinking water sterilized with steam under high pressure. During the acclimation and test periods, 7 animals per polycarbonate MI cage (26×42×18 cm; manufactured by Myoung-jin Mechanical Co., Korea) were raised.

The raised mice were measured for body weight before-test and randomly divided into the following three groups with no difference in body weight therebetween: a control group consisting of 7 animals; a group consisting of 8 animals immediately administered with the multipotent stem cells; and a group consisting of 3 animals administered with the multipotent stem cells after one day (see Table 1).

TABLE 1
Construction of test group

| | | Numbers | |
|---|---|---|---|
| Groups | Sex | of animal | # |
| Control group | female | 7 | CF-1˜CF-7 |
| Immediately | female | 8 | MSCF-1˜MSCF-8 |
| administered group | | | |
| with the multipotent | | | |
| stem cells | | | |
| Administered group | female | 3 | MSC-1F1-1˜MSC-1F-3 |
| with the multipotent | | | |
| stem cells after | | | |
| one day | | | |

According to the prior article (Nicholson, C. D. et al., Int. J. Sports Med., 13(1):60, 1992), the three animal groups were anesthetized by intraabdominal injection of 1.2 mg ketamine, and then, the left femoral region of each individual was incised, and the femoral artery was severed and sutured to establish a mouse model with Buerger's disease.

The multipotent stem cells cultured in Example 1 were washed two times with PBS and treated with 0.25% trypsin, and the cells were collected and centrifuged to remove the supernatant. Then, after inactivating trypsin by washing with PBS, the cells were subjected to second centrifugation to collect 1.3×10⁶˜10⁷ cells. The collected cells were suspended in 100 μl of sterilized saline and administered into the femoral muscle of each member of the animal group immediately administered with the multipotent stem cells. Also, 24 hours after the induction of ischemic necrosis, 1.3×10⁶˜10⁷ multipotent stem cells were suspended in 100 μl of sterilized saline, and administered into the left femoral muscle of each member of the test animal group administered with the multipotent stem cells after one day.

(2) Observation of Symptom Days and Amputation Days for Ischemic Necrotic Model

During 30 days after the administration of the inventive multipotent stem cells to the mice with ischemic necrosis induced as described above, the femoral regions of the test animals were observed. Days showing symptoms and days showing amputation were separately recorded, and before the initiation of test and at the end of test, the test animals were measured for body weight and subjected to autopsy. For statistical analysis on the test animal's body weight and the like measured during the test, one-way ANOVA was carried out to examine the significance between the groups, and when the significance was acknowledged, Dunnett's t-test was carried out to examine the statistical significance between the control group and the test groups (p<0.05).

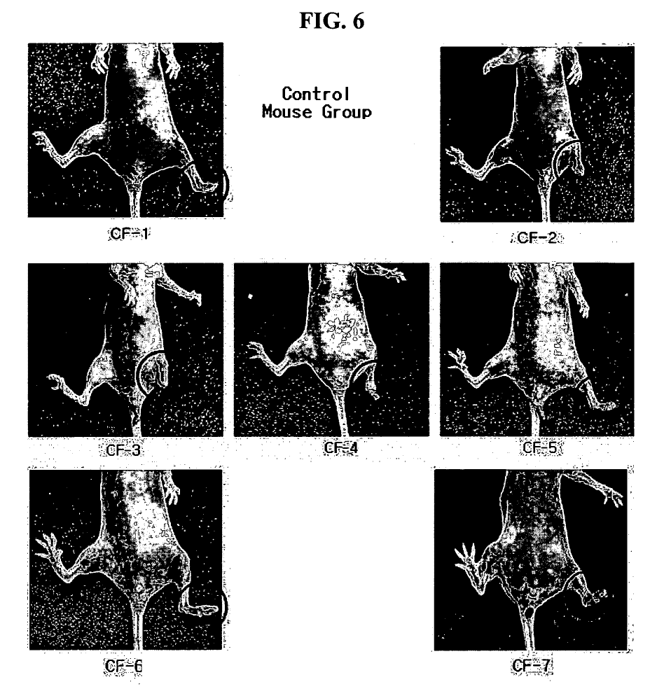

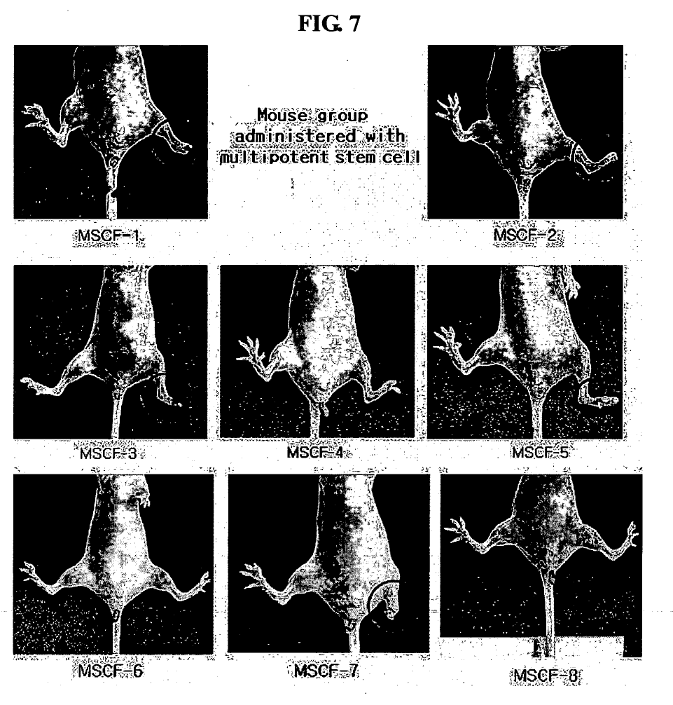

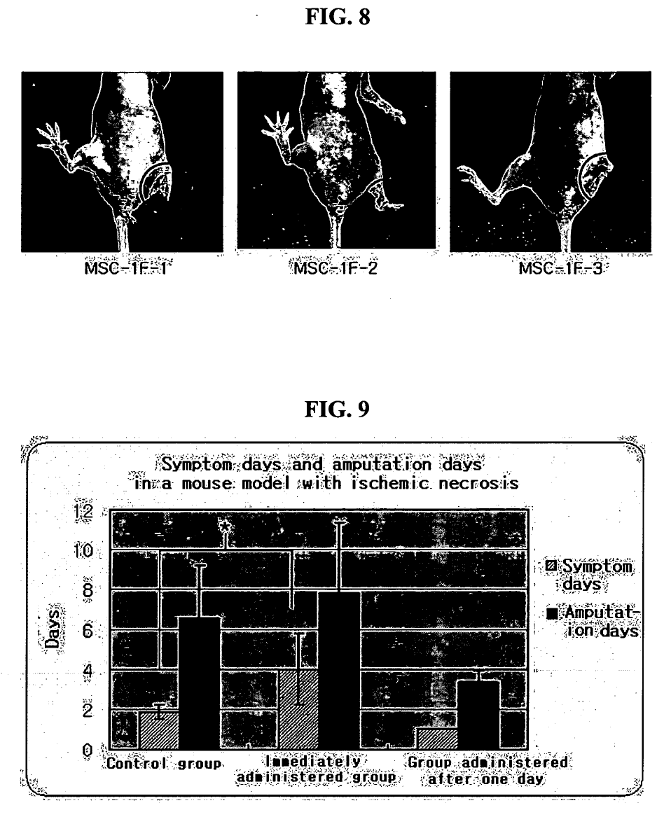

As a result, as shown in FIGS. 6 to 8, all the members of the control group untreated with the inventive umbilical cord blood-derived multipotent stem cells showed a cut phenomenon (observed in a red circle) after 30 days after the severing of blood vessels (see FIG. 6). On the other hand, the group immediately administered with the multipotent stem cells showed an amputation event in only five animals among a total of 8 animals, and individual Nos. 6 and 8 showed almost normal behavior patterns even if they had caused Buerger's disease, and individual No. 4 was prevented from amputation caused by Buerger's disease (see FIG. 7). Meanwhile, the group administered with the multipotent stem cells after one day showed the occurrence of amputation events in all the three test animals, suggesting that this group has little or no therapeutic effect on the treatment of angiogenesis as compared to the group administered immediately with the multipotent stem cells (see FIG. 8).

Furthermore, on the basis of the day when the ischemic necrosis model was established, each individual was examined for days showing symptoms (feeling cold at severed vascular regions and showing a Raynaud's phenomenon) and for days showing the amputation of the left foot or toe (Table 2).

TABLE 2

| | | | | | Symptom | Amputation |
|---|---|---|---|---|---|---|
| | | Weight before | Weight after | No. of amputated | days ± | days ± |
| | No. of | experiment | experiment | animal | standard | standard |
| Groups | animal | (g) ± SD | (g) ± SD | (amputation rate) | deviation | deviation |
| Control group | 7 | 20.0 ± 0.89 | 20.2 ± 1.07 | 7 (100%) | 1.86 ± 0.38 | 6.57 ± 2.64 |
| Immediately administered | 8 | 20.0 ± 0.74 | 20.4 ± 0.55 | 5 (62.5%) | 4.00 ± 1.77 | 7.80 ± 3.56 |
| group with the | | | | | | |
| multipotent stem cells | | | | | | |
| Administered group with | 3 | 20.1 ± 1.27 | 20.4 ± 1.78 | 3 (100%) | 1.00 ± 0 | 3.33 ± 0.58 |
| the multipotent stem | | | | | | |
| cells after one day | | | | | | |

As a result, as shown in Table 2, the group immediately administered with the multipotent stem cells showed a significance of less than 0.05 in the symptom days and the amputation days, and the remaining groups did not show significance. The body weight of each individual was measured and calculated for the average and standard deviation of each group, and as a result, a significant change in body weights measured before and after the test could not be observed.

Meanwhile, the symptom days and the amputation days were compared between the control group and the groups administered with the inventive umbilical cord blood-derived multipotent stem cells (FIG. 9). As a result, the group immediately administered with the multipotent stem cells showed a statistically significant change of less than 0.05 in the symptom days as compared to the control group. Although there was no significant change in the amputation days, the group immediately administered with the multipotent stem cells was longer in the amputation days than the control group and the group administered with the multipotent stem cells after one day, indicating that an amputation event by vascular severance occurs slowly in the group immediately administered with the multipotent stem cells.

Amputation rate for each group with ischemic necrosis was examined, and as a result, as shown in FIG. 10, the control group and the group administered with the multipotent stem cells after one day showed an amputation rate of 100%, whereas the group administered immediately with the multipotent stem cells showed an amputation rate of 62.5%. These results suggest that the administration of the multipotent stem cells immediately after vascular severance allows an amputation event to be reduced and also advantageously acts to lower an amputation line.

(3) Angiography

Angiography is a vascular evaluation using X-ray and makes it possible to see blood vessels on X-rays by injecting a contrast agent (iodine-113) into the heart's blood vessels of mice. The presence or absence of abnormality in the blood vessels is examined on X-rays to able to determine the name of disease, the location of lesions, or the progression of disease.

Before autopsy, each individual of the control group, the group immediately administered with the multipotent stem cells, and the group administered with the multipotent stem cells after one day, was anesthetized by intraabdominal injection with ketamine (100 mg/kg), and administered with a vascular contrast agent. After 2 minutes, each group was observed for femoral arteries and new blood vessels using X-rays.

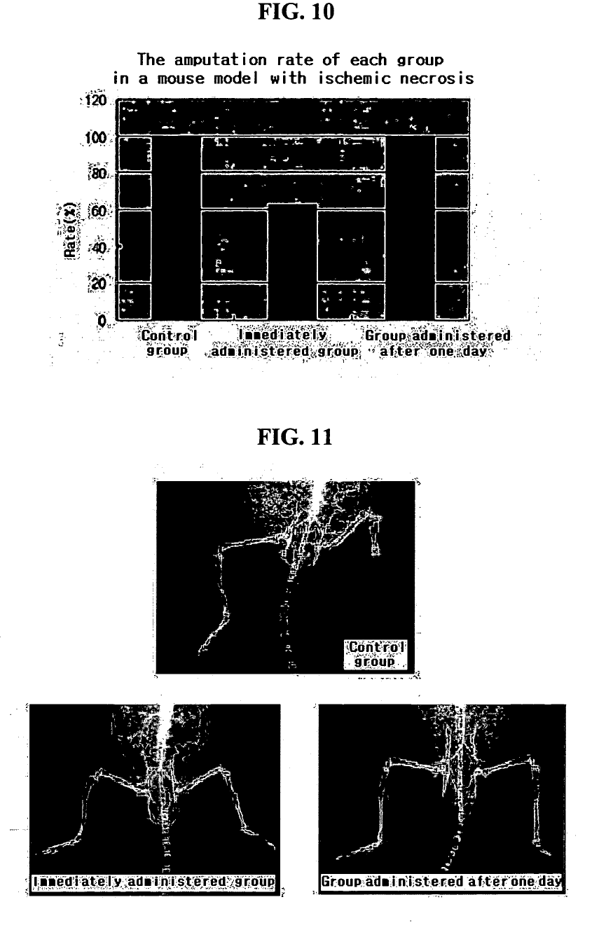

As a result, as shown in FIG. 11, new blood vessels could not be observed in the control group and the group administered with the multipotent stem cells after one day, but could be observed in the group immediately administered with the multipotent stem cells, where the amputation event of the left lower limb did not occur.

(4) In Situ Hybridization

Upon autopsy, the muscular blood of all the individuals of each group was removed by the collection of heart blood. Muscular tissues in the left and right femoral regions of each individual of the animal groups were fixed with a fixing solution consisting of a mixture of 4% paraformaldehyde phosphate solution and 1.5% sucrose solution. The fixed femoral tissues were left to stand at 4° C. until they settled in 30% sucrose phosphate solution. Then, each of the tissues was embedded in paraffin and finely sectioned with a tissue microtome in a thickness of 5 μm. Then, the sections were reacted with prehybridization solution (50% formamide, 4×SSC, 50 mM DDT, 4× Denhart's solution, ×TED, 100 μg/ml denatured salmon sperm DNA, 250 μg/ml yeast RNA) at 42° C. for 1 hour. To the resulting solution, DIG-labeled DNA (100 ng/ml) was added and reacted with the prehybridization solution for 24 hours so that a DIG labeled human-specific DNA probe was bound to mRNA. The femoral tissues were washed with each of 2×, 1× and 0.5×SSC solutions two times for 10 minutes each time, and fixed on a slide glass, followed by drying at room temperature for 2 hours.

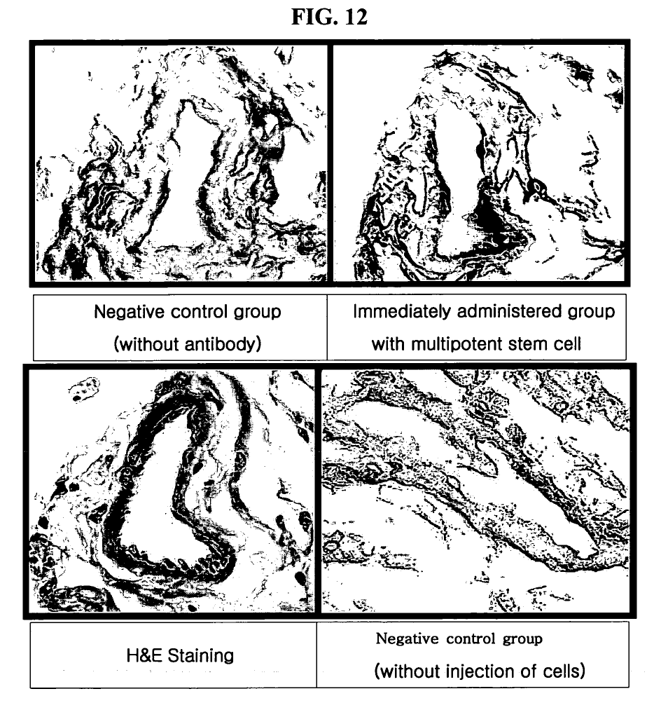

As a result, as could be seen in FIG. 12, the control group was not labeled with the human probe, but the individuals of the group administered with the multipotent stem cells were labeled with the human probe in endothelial cells.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described in detail above, the present invention provides multipotent stem cells isolated from umbilical cord blood using human serum or plasma. The stem cells according to the present invention have ability to differentiate into osteogenic cells, nerve cells and the like, even if they are adult stem cells. Thus, the inventive stem cells will be useful as a cellular therapeutic agent for ischemic necrotic diseases caused by occlusive or ischemic arterial disease.

1. Adult stem cells which are obtained by culturing umbilical cord-derived blood in a medium containing 5-20% of human serum or plasma and show the characteristics of:
(a) showing positive immunological responses to all of CD24, CD29, CD31, CD33, CD45 and CD49B, and negative immunological responses to CD34, CD51/61, CD62L, CD62P, CD73, CD90, CD133 and CD135;
(b) growing adhered to plastics and showing round-shaped or spindle-shaped morphological features; and
(c) having ability to differentiate into mesodermal, endodermal and ectodermal cells.

2. The adult stem cells according to claim 1, wherein additionally show positive immunological responses to SH-2 and/or SH-3.

3. The adult stem cells according to claim 1, showing positive or negative immunological responses to CD44, CD105 and CD117.

4. The adult stem cells according to claim 1, wherein the mesodermal cells are osteogenic cells, nerve cells or endothelial cells.

5. A method for producing adult stem cells showing the following characteristics:
(a) showing positive immunological responses to all of CD24, CD29, CD31, CD33, CD45 and CD49B, and negative immunological responses to CD34, CD51/61, CD62L, CD62P, CD73, CD90, CD133 and CD135;
(b) growing adhered to plastics and showing round-shaped or spindle-shaped morphological features; and
(c) having ability to differentiate into mesodermal, endodermal and ectodermal cells, the method comprising the steps of: culturing umbilical cord-derived blood in a medium containing 5-20% of human serum or plasma; and recovering the adult stem cells.

6. The method for producing adult stem cells according to claim 5, wherein the human serum or plasma is autologous or allologous.

7. A cellular therapeutic agent for ischemic necrotic disease caused by occlusive arterial disease, which contains the adult stem cells according to claim 1, as active ingredients.

8. The cellular therapeutic agent for ischemic necrotic disease caused by occlusive arterial disease according to claim 7, wherein the ischemic necrotic disease is myocardial infarction, cerebral infarction, Buerger's disease, or gangrene of extremities resulting from diabetic complications.

9. A cellular therapeutic agent for ischemic necrotic disease caused by occlusive arterial disease, which contains the adult stem cells according to claim 2, as active ingredients.

10. A cellular therapeutic agent for ischemic necrotic disease caused by occlusive arterial disease, which contains the adult stem cells according to claim 3, as active ingredients.

11. A cellular therapeutic agent for ischemic necrotic disease caused by occlusive arterial disease, which contains the adult stem cells according to claim 4, as active ingredients.

12. A method of treatment of cardiovascular disease in an individual experiencing same, said method comprising administration to said individual of a therapeutic amount of adult stem cells as claimed in claim 1.

13. The method of claim 12, wherein said cardiovascular disease comprises occlusive arterial disease.

14. The method of claim 12, wherein said cardiovascular disease comprises myocardial disease.

15. A method of treatment of necrosis involving a limb of an individual experiencing same, said method comprising administration to said individual of a therapeutic amount of adult stem cells as claimed in claim 1.

16. A cellular therapeutic composition comprising the adult stem cells of claim 1 as active ingredients.
